(19) United States
(12) Patent Application Publication
(10) Pub. No.: US 2006/0161163 A1
(43) Pub. Date: Jul. 20, 2006

(54) Instrument for reconstructing ligament and method of reconstructing ligament

(76) Inventor: Konsei Shino, Suita-shi (JP)

Correspondence Address: NIXON & VANDERHYE, PC, 901 NORTH GLEBE ROAD, 11TH FLOOR, ARLINGTON, VA, 22203, US

(21) Appl. No.: 10/563,913

(22) PCT Filed: Jul. 8, 2004
(86) PCT No.: PCT/JP04/09739

§ 371 Date: Jan. 10, 2006

(30) Foreign Application Priority Data
Jul. 11, 2003 (JP) 2003-196051

(51) Int. Cl. A61F 5/00

(52) U.S. Cl. 606/86

(57) ABSTRACT

A dilator (1) for reconstructing an anterior cruciate ligament, comprising a tip part (4) having two through-holes (2) and (3) arranged in juxtaposition and parallel with each other, a body part (5), and a rear end part (8) having two through-holes (6) and (7) arranged in juxtaposition and coaxially with the two through-holes (2) and (3), respectively. The tip part (4) and the body part (5) are formed in uniform generally elongated shapes in cross section longer in the arranged direction of the through-holes (2) and (3) so that a flat tunnel can be formed by striking the instrument from the rear end part (8) thereof to drive the tip part (4) and the body part (5) into an articular bone part. Thus, the anterior cruciate ligament can be reconstructed to a more tough ligament similar to a normal one in a shorter time.

### TECHNICAL FIELD

The present invention relates to a ligament reconstruction device and a ligament reconstruction method. More specifically, the invention relates to a ligament reconstruction device and a ligament reconstruction method to be utilized for forming a socket in an articular bone for fixating one end portion of a ligament when a damaged ligament such as an anterior cruciate ligament (a ligament connecting a femur and a tibia) is reconstructed.

### BACKGROUND ART

In conventional anterior cruciate ligament reconstruction (technique), for example, a cylindrical bone tunnel having a round cross section is formed in a tibia as extending through a bone wall of the tibia, and another cylindrical bone tunnel having a round cross section is formed in a femur as extending through a bone wall of the femur. A looped ligament graft is inserted through these bone tunnels with one end portion thereof fixated to the femur via a metal device. A body of a hollow cylindrical metal screw is inserted into the bone tunnel of the tibia from an open end of the bone tunnel (see Japanese Unexamined Patent Publication No. 2001-25478).

The anterior cruciate ligament reconstruction technique has been steadily developed. This technique is applied to an increased number of reconstruction operations, and makes it possible to properly perform the operations with a reduced rate of occurrence of complications. Therefore, the anterior cruciate ligament reconstruction is now a standard treatment with an excellent treatment effect.

Young and healthy athletes often need the anterior cruciate ligament reconstruction. Therefore, it is constantly demanded to reconstruct a more robust ligament in a shorter period of time.

### DISCLOSURE OF THE INVENTION

As a result of intensive studies in view of the foregoing, the inventor of the present invention has found that there is a great gap between a ligament graft having a generally rectangular or elliptical cross section (e.g., having a minor axis of 4 mm and a major axis of 8 mm) rather than a round cross section and an interior surface of a conventionally formed round bone tunnel (e.g., having an inner diameter of 8 to 10 mmφ), and reduction of the gap facilitates the bonding of the ligament graft to a bone. Thus, the present invention has been attained.

According to the present invention, there is provided a ligament reconstruction device, which comprises a tip portion having two parallel through-holes formed therein in juxtaposition, a rear end portion having two through-holes formed therein in juxtaposition coaxially with the two through-holes of the tip portion, and a connection portion connecting the rear end portion and the tip portion and having a single connection hole connecting one of the through-holes of the tip portion coaxially to one of the through-holes of the rear end portion, the connection portion being thinner and longer than the tip portion and the rear end portion. The tip portion has a generally elliptical or rectangular cross section elongated in a direction in which the through-holes thereof are juxtaposed, so that the tip portion is driven into an articular bone to form a flat socket in the bone by hitting the rear end portion.

According to the present invention, the tip portion has a generally elliptical or rectangular cross section rather than a simple round cross section. Therefore, a bone cavity to be formed in the bone has a cross section close to the cross section of an ordinary ligament, so that the outer peripheral surface of the ligament graft is entirely brought into proximity to the interior surface of the bone cavity. Hence, the ligament can be bonded to the bone with a greater contact area in a shorter period of time.

The connection portion connecting the tip portion and the rear end portion is thinner and longer than the tip portion. Therefore, the ligament reconstruction device can be easily inserted into a gap between a pair of bones of a joint (e.g., a gap between a femur and a tibia of a knee joint), while avoidance of damage to surrounding soft tissue during insertion or retrieval of the device.

According to another aspect of the present invention, there is provided a ligament reconstruction method utilizing the aforesaid ligament reconstruction device, the method comprising the steps of: drilling a guide pin into an articular bone; fitting the guide pin in two of the through-holes and the connection hole of the ligament reconstruction device aligned with each other, and drilling another guide pin into the articular bone through the other two through-holes of the ligament reconstruction device; removing portions of the bone around the previously-inserted two guide pins by over-drilling; and driving the tip portion of the ligament reconstruction device into the articular bone toward a lateral cortex of the articular bone by hitting the rear end portion of the ligament reconstruction device with the two guide pins respectively fitted in the two through-holes and the connection hole of the ligament reconstruction device aligned with each other and in the other two through-holes of the ligament reconstruction device to form a flat socket into which one end portion of a ligament is to be inserted.

### BRIEF DESCRIPTION OF THE DRAWINGS

FIG. 1 is a plan view illustrating a ligament reconstruction device according to one embodiment of the present invention;

FIG. 2 is a right side view of the ligament reconstruction device shown in FIG. 1;

FIG. 3 is a sectional view taken along a line A-A in FIG. 2;

FIG. 4 is an enlarged partial rear view of the ligament reconstruction device shown in FIG. 1;

FIG. 5 is an explanatory diagram illustrating a ligament reconstruction method utilizing the ligament reconstruction device shown in FIGS. 1 to 4;

FIG. 6 is an explanatory diagram illustrating the next step of the ligament reconstruction method utilizing the ligament reconstruction device;

FIG. 7 is an explanatory diagram illustrating the next step of the ligament reconstruction method utilizing the ligament reconstruction device;

FIG. 8 is an explanatory diagram illustrating the next step of the ligament reconstruction method utilizing the ligament reconstruction device;

FIG. 9 is an explanatory diagram illustrating the next step of the ligament reconstruction method utilizing the ligament reconstruction device; and

FIG. 10 is an explanatory diagram illustrating the next step of the ligament reconstruction method utilizing the ligament reconstruction device.

### BEST MODE FOR IMPLEMENTING THE INVENTION

A ligament reconstruction device according to the present invention mainly includes a tip portion, a rear end portion and a connection portion which connects the tip portion and the rear end portion.

The tip portion has two parallel through-holes formed therein in juxtaposition, and has a generally elliptical or rectangular cross section elongated in a direction in which the two through-holes are juxtaposed.

The generally elliptical or rectangular cross section preferably has a major axis/minor axis ratio of 2 to 5. The elliptical cross section is preferably of a generally oval shape or a racetrack-like elliptical shape. The racetrack-like elliptical shape is preferably defined by a pair of parallel straight lines spaced a distance of 3 to 6 mm from each other and each having a length of 4 to 8 mm and a pair of semicircles connecting opposite ends of these straight lines. The rectangular cross section preferably has a minor edge length of 3 to 6 mm and a major edge length of 7 to 14 mm. Therefore, the tip portion preferably has a cross sectional area of 21 to 84 mm².

The tip portion preferably has a length of 5 to 10 mm.

The connection portion has a single connection hole which connects one of the through-holes of the tip portion parallel to one of the through-holes of the rear end portion, and is thinner and longer than the tip portion and the rear end portion. Therefore, the connection portion can be easily inserted into a gap between a pair of articular bones (e.g., a gap between a femur and a tibia of a knee joint), and prevents damage to surrounding soft tissue during attachment and detachment of the device. The connection portion preferably has a generally round or oval cross section so as not to damage the surrounding soft tissue.

The rear end portion has two through-holes which are respectively coaxially with the two through-holes of the tip portion, respectively, and is typically of a thick elliptical or round disk shape having a greater cross sectional area than the tip portion. The rear end portion is hit by a hummer to drive the tip portion into the articular bone. Thus, a flat socket having a cross section conformable to the cross section of the tip portion is formed in the articular bone.

Next, an exemplary method for ligament reconstruction to be performed with the use of the ligament reconstruction device having the aforesaid construction will be described.

(1) A guide pin is first drilled into the articular bone.

(2) With the guide pin fitted in two of the through-holes and the connection hole of the ligament reconstruction device aligned with each other, another guide pin is stuck into the articular bone through the other two parallel through-holes of the ligament reconstruction device.

(3) Portions of the bone stuck with the guide pins are over-drilled.

(4) With the two guide pins respectively fitted in the two through-holes and the connection hole of the ligament reconstruction device aligned with each other and in the other two through-holes of the ligament reconstruction device, the rear end portion of the ligament reconstruction device is hit by a hummer to drive the tip portion of the ligament reconstruction device into the articular bone toward a lateral cortex of the articular bone. Thus, a flat socket into which one end portion of a ligament is to be inserted is formed as having a cross section conformable to the cross section of the tip portion.

The flat socket desirably has a depth of 10 to 23 mm. Where the ligament reconstruction is reconstruction of an anterior cruciate ligament, the articular bone is a femur. An artificial ligament may be used as the ligament, but a ligament graft, particularly a patellar tendon of a human (a patient or a dead person) with a bone piece, is preferred.

An embodiment of the present invention will hereinafter be described in detail with reference to the attached drawings.

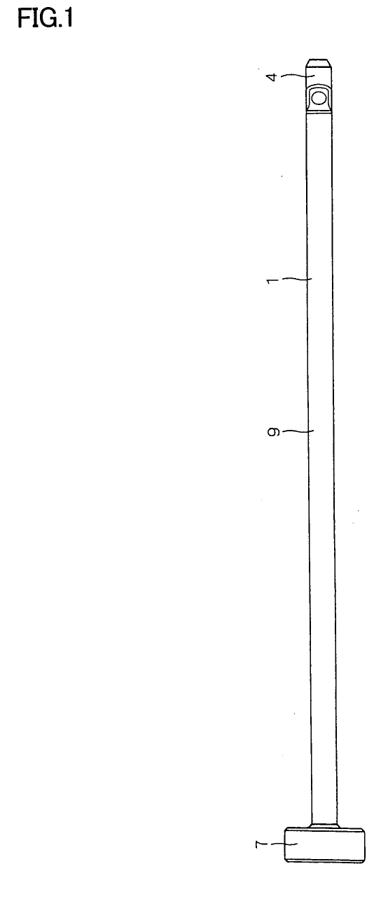

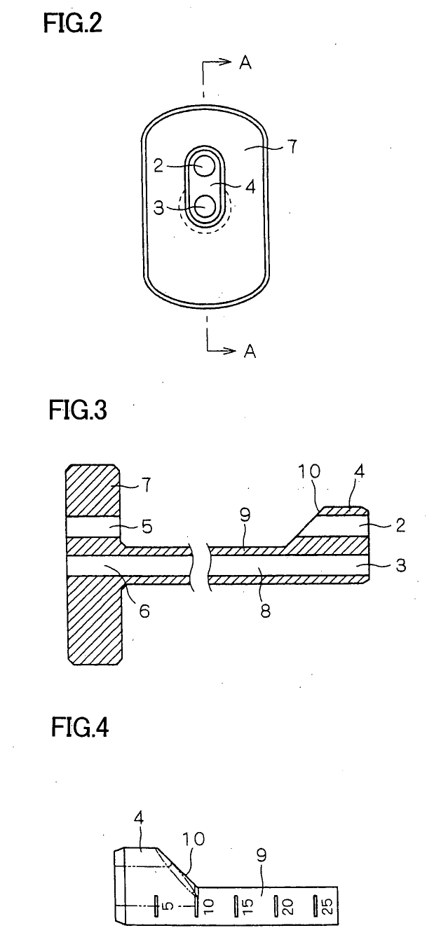

Referring first to FIGS. 1 to 3, an anterior cruciate ligament reconstruction dilator 1 as the inventive ligament reconstruction device includes a tip portion 4 having two parallel through-holes 2, 3 formed therein in juxtaposition, a rear end portion 7 having two through-holes 5, 6 formed therein parallel to the two through-holes 2, 3, and a connection portion 9 which connects the rear end portion 7 and the tip portion 4 and has a single connection hole 8 connecting the through-holes 3, 6 parallel to each other. The connection portion 9 is thinner and longer than the tip portion 4 and the rear end portion 7.

The tip portion 4 has an elliptical cross section which is elongated in a direction in which the through-holes 2, 3 are juxtaposed. More specifically, the tip portion 4 has a generally racetrack-shaped elliptical cross section (like an athletic racetrack). The rear end portion 7 has a generally racetrack-shaped elliptical cross section which is larger than the cross section of the tip portion. The connection portion 9 has a generally round cross section having a smaller area.

The through-holes 2, 3, 5, 6 and the connection hole 8 each have an inner diameter of 2.5 mmφ. Straight lines partly defining the cross section of the tip portion 4 are spaced a distance of 5 mm from each other, and each have a length of 6 mm. The cross section of the connection portion 9 has an outer diameter of 5 mmφ. Straight lines partly defining the cross section of the rear end portion 7 are spaced a distance of 16 mm from each other, and each have a length of 18 mm.

A rear opening face 10 of the through-hole 2 of the tip portion 4 is inclined at about 45 degrees. Therefore, the anterior cruciate ligament reconstruction dilator 1 can be easily removed from a joint, and prevents damage to surrounding soft tissue. As shown in FIG. 4, scale marks of 5, 10, 15, . . . (mm) which indicate the depth of a flat socket to be formed are provided in the vicinity of the tip portion 4.

An anterior cruciate ligament reconstruction method to be performed with the use of the anterior cruciate ligament reconstruction dilator 1 having the aforesaid construction will next be described with reference to FIGS. 5 to 10.

(1) A first guide pin 11 having a diameter of 2.4 mmφ is drilled into a femur D toward a lateral cortex of the femur D from the center of a portion of the femur D (a 9 o'clock position of a right knee, a 9 o'clock position of a left knee or a position 6 mm apart from a posterior end of an intercondylar notch) to which posterolateral fibers of an anterior cruciate ligament are bonded (see FIG. 5). A reference character K denotes a tibia.

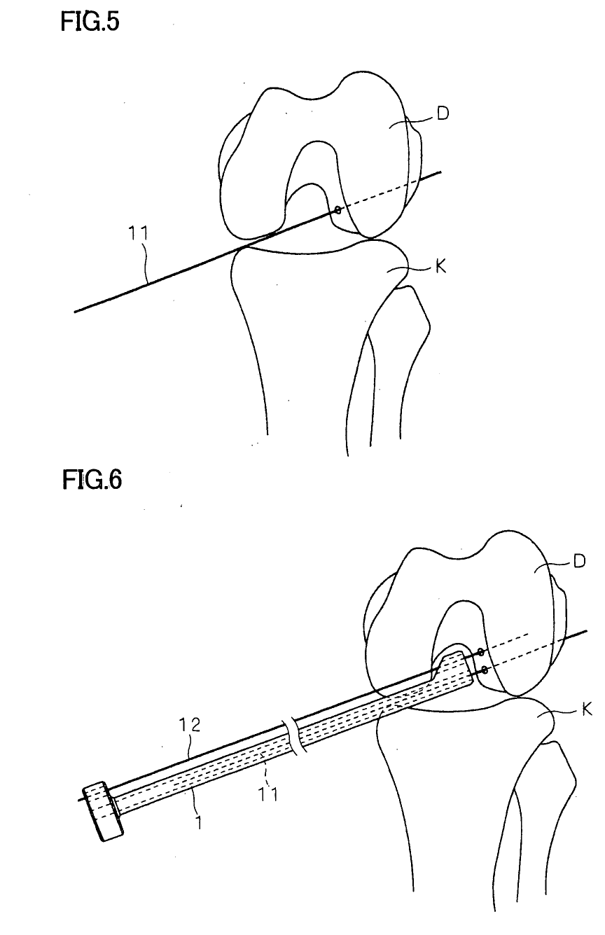

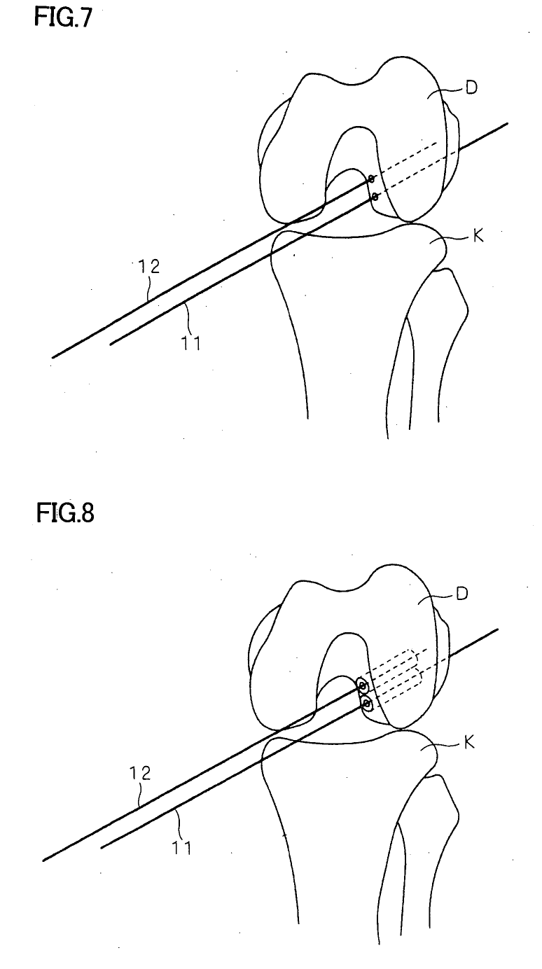

(2) With the guide pin 11 fitted in the two through-holes 3, 6 and the connection hole 8 of the anterior cruciate ligament reconstruction dilator 1 aligned with each other, a second guide pin 12 having a diameter of 2.4 mmφ is drilled into the femur D through the two through-holes 2, 5 of the anterior cruciate ligament reconstruction dilator 1 (see FIGS. 6 and 7).

(3) The femur is over-drilled along the guide pins 11, 12 to a depth of about 20 mm from the inside of a joint by a 5-mmφ cannulated drill (FIG. 8).

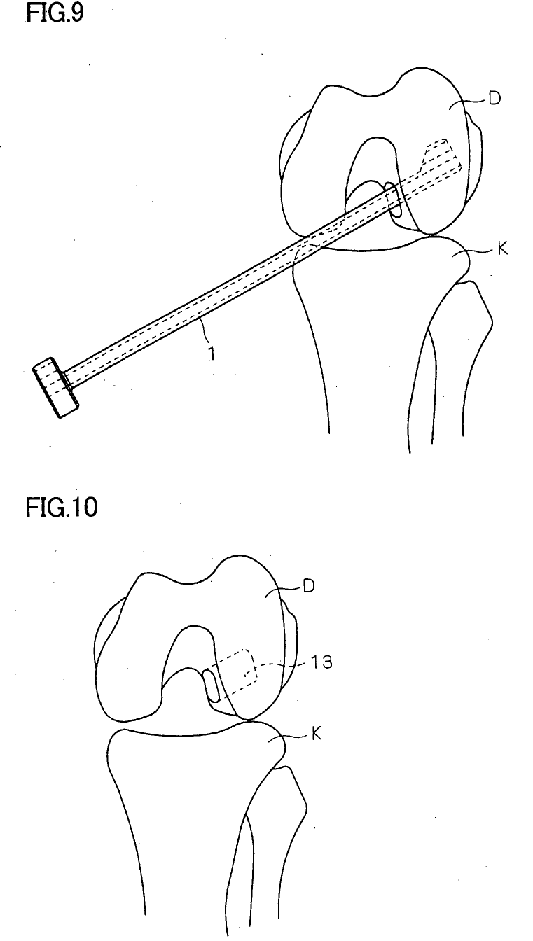

(4) With the two guide pins 11, 12 fitted in the through-holes and the connection hole of the anterior cruciate ligament reconstruction dilator 1, the rear end portion 7 of the anterior cruciate ligament reconstruction dilator 1 is hit by a hummer to drive the tip portion 4 into the femur D toward a lateral cortex of the femur D. Thus, a flat socket 13 of a generally rectangular parallelepiped shape having width×length×depth of 5 mm×10 mm×20 mm is formed (see FIGS. 9 to 10).

One end portion of a patellar tendon with a bone piece is inserted into the flat socket 13 thus formed and properly fixated in the flat socket 13 by a conventional method.

Thus, the patellar tendon with the bone plug is firmly bonded to the femur D in a short period of time (e.g., in 28 days).

According to the present invention, as described above, the tip portion has a generally elliptical or rectangular cross section rather than a simple round cross section. Therefore, a bone cavity to be formed in a bone has a cross section close to the cross section of an ordinary ligament graft, so that the outer peripheral surface of the ligament graft is entirely brought into proximity to the interior surface of the bone cavity. Hence, the ligament graft can be bonded to the bone with a greater contact area in a shorter period of time.

The connection portion connecting the tip portion and the rear end portion is thinner and longer than the tip portion. Therefore, the inventive ligament reconstruction device can be easily inserted into a gap between a pair of articular bones (e.g., a gap between a femur and a tibia of a knee joint), while avoiding damage to surrounding soft tissue during insertion or retrieval of the device.

1. A device for ligament reconstruction comprising:
a tip portion having two parallel through-holes formed therein in juxtaposition;
a rear end portion having two through-holes formed therein in juxtaposition coaxially with the two through-holes of the tip portion; and
a connection portion connecting the rear end portion and the tip portion and having a single connection hole connecting one of the through-holes of the tip portion coaxially to one of the through-holes of the rear end portion, the connection portion being thinner and longer than the tip portion and the rear end portion,
wherein the tip portion has a generally elliptical or rectangular cross section elongated in a direction in which the through-holes thereof are juxtaposed, so that the tip portion is driven into an articular bone to form a flat socket in the bone by hitting the rear end portion.

2. A ligament reconstruction device as set forth in claim 1, wherein the generally elliptical or rectangular cross section has a major axis/minor axis ratio of 2 to 5.

3. A ligament reconstruction device as set forth in claim 1, wherein the elliptical cross section is of a generally oval shape or a racetrack-like elliptical shape.

4. A ligament reconstruction device as set forth in claim 3, wherein the racetrack-like elliptical shape is defined by a pair of parallel straight lines spaced a distance of 3 to 6 mm from each other and each having a length of 4 to 8 mm and a pair of semicircles connecting opposite ends of the straight lines.

5. A ligament reconstruction device as set forth in claim 1, wherein the rectangular cross section has a minor edge length of 3 to 6 mm and a major edge length of 7 to 14 mm.

6. A ligament reconstruction device as set forth in claim 1, wherein the tip portion has a cross sectional area of 21 to 84 mm².

7. A ligament reconstruction device as set forth in claim 1, wherein the tip portion has a length of 5 to 10 mm.

8. A ligament reconstruction device as set forth in claim 1, wherein the connection portion has a generally round or oval cross section.

9. A ligament reconstruction device as set forth in claim 1, wherein the ligament reconstruction is reconstruction of an anterior cruciate ligament graft.

10. A method for ligament reconstruction utilizing a ligament reconstruction device as recited in claim 1, the method comprising the steps of:
drilling a guide pin into an articular bone;
fitting the guide pin in two of the through-holes and the connection hole of the ligament reconstruction device aligned with each other, and drilling another guide pin into the articular bone through the other two through-holes of the ligament reconstruction device;
removing portions of the bone around the previously-inserted two guide pins by over-drilling; and
driving the tip portion of the ligament reconstruction device into the articular bone toward a lateral cortex of the articular bone by hitting the rear end portion of the ligament reconstruction device with the two guide pins respectively fitted in the two through-holes and the connection hole of the ligament reconstruction device aligned with each other and in the other two through-holes of the ligament reconstruction device to form a flat socket into which one end portion of a ligament graft is to be inserted.

11. A ligament reconstruction method as set forth in claim 10, wherein the flat socket has a depth of 10 to 23 mm.

12. A ligament reconstruction method as set forth in claim 10, wherein the ligament graft is an anterior cruciate ligament graft with a bone piece.

13. A ligament reconstruction method as set forth in claim 10, wherein the ligament reconstruction is reconstruction of an anterior cruciate ligament graft, and the articular bone is a femur.
